(19) United States
(12) Patent Application Publication
(10) Pub. No.: US 2023/0392143 A1
(43) Pub. Date: Dec. 7, 2023

(54) METHODS FOR PREPARING A SEQUENCING LIBRARY FROM SINGLE-STRANDED DNA

(71) Applicant: GRAIL, LLC, Menlo Park, CA (US)

(72) Inventors: Farnaz Absalan, Menlo Park, CA (US); Gordon Cann, Menlo Park, CA (US); Arash Jamshidi, Menlo Park, CA (US)

(21) Appl. No.: 18/453,973

(22) Filed: Aug. 22, 2023

Related U.S. Application Data
(63) Continuation of application No. 16/647,419, filed on Mar. 13, 2020, now abandoned.
(60) Provisional application No. 62/558,760, filed on Sep. 14, 2017.

(51) Int. Cl. C12N 15/10; C12Q 1/6874; C12Q 1/6886

(52) U.S. Cl. CPC C12N 15/1068; C12Q 1/6886; C12Q 1/6874

(57) ABSTRACT

Methods for generating a sequencing library from a sample comprising a plurality of single-stranded DNA molecules are provided, along with methods of using the generated sequencing library for detecting cancer, determining cancer stage, monitoring cancer progression, and/or determining a cancer classification from a test sample obtained from a subject.

### CROSS-REFERENCE TO RELATED APPLICATIONS

This application is a continuation of U.S. application Ser. No. 16/647,419 filed Mar. 13, 2020, which is a 371 US National Stage Entry of International Application No. PCT/US2018/051138 filed Sep. 14, 2018, which claims priority benefit of the filing date of U.S. Provisional Patent Application No. 62/558,760, filed on Sep. 14, 2017. The entire disclosures of which are herein incorporated by reference in their entireties.

### FIELD OF THE INVENTION

The present invention relates to methods for generating a sequencing library from a sample comprising a plurality of single-stranded DNA molecules. Aspects of the invention include methods of using the generated sequencing library for detecting cancer, determining cancer stage, monitoring cancer progression, and/or determining a cancer classification from a test sample obtained from a subject.

### BACKGROUND OF THE INVENTION

Aberrant DNA methylation has been implicated in many disease processes, including cancer. DNA methylation profiling using bisulfite conversion sequencing is increasingly recognized as a valuable diagnostic tool for detection and diagnosis of cancer. The first step in bisulfite sequencing is to treat the DNA with bisulfite to convert cytosine bases to uracil. As a result of this conversion, hybridization between complementary DNA strands is disrupted, leading to single-strand DNA (ssDNA) substrates. During preparation of a sequencing library, addition of sequencing adapters to ssDNA using a ligation step is very inefficient. Accordingly, there is a need in the art for new methods of preparing sequencing libraries from ssDNA substrates.

### SUMMARY OF THE INVENTION

Aspects of the invention include methods for preparing a sequencing library from a test sample comprising a plurality of single-strand DNA fragments, the method comprising: (a) obtaining a test sample comprising a plurality of single-stranded DNA (ssDNA) fragments; (b) adding a plurality of non-templated nucleotide bases to the 3′-end of the ssDNA fragments generating a plurality of 3′-polynucleotide tailed ssDNA fragments; (c) annealing a plurality of single-stranded DNA (ssDNA) oligonucleotide adapters to the 3′-end of the polynucleotide tailed ssDNA fragments to generate a plurality of partially double-stranded DNA fragment-adapter constructs, wherein the oligonucleotide adapters comprise a region complementary to the 3′-polynucleotide tail of the tailed ssDNA fragments; (d) extending the 3′-tail of the oligonucleotide adapters using a DNA polymerase and the ssDNA fragment as a template to generate a plurality of double-stranded DNA (dsDNA) molecules; (e) ligating a double-strand DNA adapter to the plurality of dsDNA molecules obtained from step (d) to generate a plurality of dsDNA adapter-fragment constructs, wherein the double-strand DNA adapters are ligated to the end of the dsDNA molecules opposite the ssDNA oligonucleotide adapters; and (f) amplifying the dsDNA adapter-fragment constructs to generate a sequencing library.

In some embodiments, the sequencing library prepared using the methods of the present invention can be sequenced to obtain a plurality of sequence reads, and the sequence reads analyzed to detect the presence or absence of cancer, determine cancer status, monitor cancer progression, and/or determine a cancer classification. In other embodiments, the sequence reads can be analyzed to determine cancer type and/or cancer tissue of origin. In still other embodiments, the sequence reads can be analyzed to monitor disease progression, monitor therapy, and/or monitor cancer growth. In accordance with these embodiments, the cancer may comprise a carcinoma, a sarcoma, a myeloma, a leukemia, a lymphoma, a blastoma, a germ cell tumor, or any combination thereof.

### BRIEF DESCRIPTION OF THE DRAWINGS

FIG. 1 is a flow diagram illustrating a method of preparing a sequencing library from ssDNA substrates, in accordance with one embodiment of the present invention;

FIG. 2 is a flow diagram illustrating a method of preparing a sequencing library from ssDNA substrates, in accordance with another embodiment of the present invention

FIG. 3 shows pictorially some of the steps of the method of FIG. 2; and

FIG. 4 is a flow diagram illustrating a method for preparing a sequencing library from a cell-free DNA test sample for use in detecting cancer, determining cancer status, monitoring cancer progression, and/or determining a cancer classification.

### DEFINITIONS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, NY 1994), provides one skilled in the art with a general guide to many of the terms used in the present application, as do the following, each of which is incorporated by reference herein in its entirety: Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6th edition (Saunders, 2007).

All publications mentioned herein are expressly incorporated herein by reference to disclose and describe the methods and/or materials in connection with which the publications are cited.

The term “amplicon” as used herein means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26: 2150-2155 (1998), and like references.

The term “amplifying” means performing an amplification reaction. A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger polynucleotide molecule. A polynucleotide, for example, can be broken up, or fragmented into, a plurality of segments, either through natural processes, as is the case with, e.g., cfDNA fragments that can naturally occur within a biological sample, or through in vitro manipulation. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N. Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al, U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); the disclosures of which are hereby incorporated by reference herein in their entireties. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references, which are incorporated by reference herein in their entireties: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); and Becker-Andre et al, Nucleic Acids Research, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2nd Edition (Cold Spring Harbor Press, New York, 2003).

The terms “subject” and “patient” are used interchangeably herein and refer to a human or non-human animal who is known to have, or potentially has, a medical condition or disorder, such as, e.g., a cancer.

The term “sequence read” as used herein refers to nucleotide sequences read from a sample obtained from a subject. Sequence reads can be obtained through various methods known in the art.

The term “read segment” or “read” as used herein refers to any nucleotide sequences, including sequence reads obtained from a subject and/or nucleotide sequences, derived from an initial sequence read from a sample. For example, a read segment can refer to an aligned sequence read, a collapsed sequence read, or a stitched read. Furthermore, a read segment can refer to an individual nucleotide base, such as a single nucleotide variant.

The term “cell-free DNA” or “cfDNA” refers to nucleic acid fragments that circulate in a subject's body (e.g., bloodstream) and originate from one or more healthy cells and/or from one or more cancer cells.

The term “circulating tumor DNA” or “ctDNA” refers to nucleic acid fragments that originate from tumor cells or other types of cancer cells, which may be released into a subject's bloodstream as a result of biological processes, such as apoptosis or necrosis of dying cells, or may be actively released by viable tumor cells.

### DETAILED DESCRIPTION OF THE INVENTION

Aspects of the invention include methods for preparing a sequencing library from a test sample comprising a plurality of ssDNA molecules or ssDNA fragments. In one embodiment, the methods of the invention utilize a terminal transferase to add a 3′-polynucleotide tail to ssDNA molecules or fragments. For example, the present invention may utilize a terminal transferase and a reaction mixture comprising one or more deoxynucleotide triphosphates (dNTP) to catalyze the addition of a poly-dNTP tail to the 3′-end of the ssDNA fragments. In accordance with the invention, the terminal transferase reaction mixture may further comprise one or more blocking nucleotides, incorporation of which terminates the 3′-tail extension by the terminal transferase, thereby controlling the length of the 3′-tail. In one example, a terminal transferase reaction mixture may comprise one or more deoxynucleotide triphosphates (dNTPs) and one or more dideoxynucleotide triphosphates (ddNTPs).

In one embodiment, a method in accordance with embodiments of the invention is used for preparing a library for methylation sequencing (e.g., after bisulfite conversion) of circulating cell-free DNA (cfDNA) to determine DNA methylation profiles that may be indicative of cancer status.

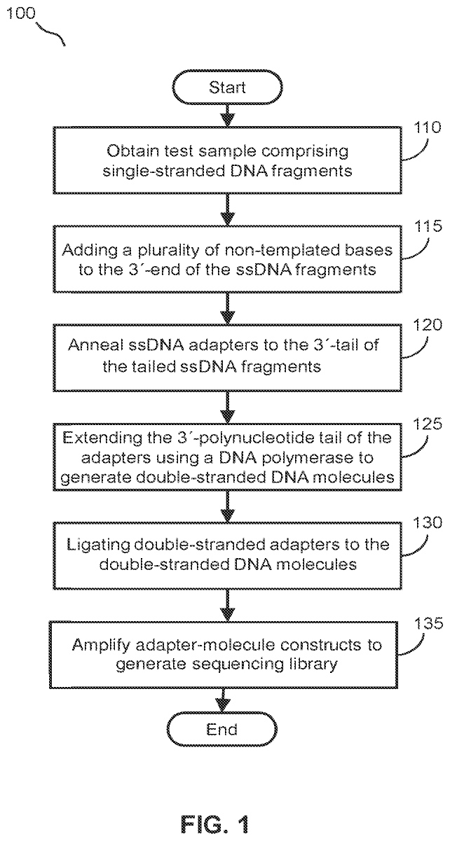

FIG. 1 illustrates a flow diagram of a method 100 for preparing a sequencing library from ssDNA molecules or fragments, in accordance with one embodiment of the present invention. In accordance with this embodiment, the method includes, but is not limited to, the following steps.

At step 110 a biological test sample is obtained comprising a plurality of ssDNA fragments. In one embodiment, the biological test sample may be a sample selected from the group consisting of blood, plasma, serum, urine and saliva samples. In other embodiments, the sample is a plasma sample from a cancer patient, or a patient suspected of having cancer. Alternatively, the biological sample may comprise a sample selected from the group consisting of whole blood, a blood fraction, a tissue biopsy, pleural fluid, pericardial fluid, cerebral spinal fluid, and peritoneal fluid. In accordance with some embodiments, the biological test sample comprises a plurality of cell-free nucleic acids (e.g., cell-free DNA (cfDNA)) fragments originating from healthy cells and from cancer cells. In one example, the ssDNA sample is a bisulfite-converted DNA sample for methylation profiling of cfDNA. Optionally, in one embodiment, cell-free nucleic acids (e.g., cfDNA) can be extracted and/or purified from the biological test sample before proceeding with subsequent library preparation steps. In general, any known method in the art can be used to extract and purify cell-free nucleic acids from the biological test sample. For example, cell-free nucleic acids can be extracted and purified using one or more known commercially available protocols or kits, such as the QIAamp circulating nucleic acid kit (Qiagen) or MagMAX Cell-Free DNA Isolation kit (Thermo Fisher Scientific).

At step 115, a polynucleotide tail is added to the 3′-ends of the ssDNA fragments. For example, terminal transferase (TdT) and a reaction mixture of a deoxynucleotide triphosphates (dNTP) is used to add a poly-dNTP tail to the 3′-ends of the ssDNA fragments. In one embodiment, the reaction mixture may further comprise one or more blocking nucleotide (e.g., one or more dideoxynucleotide triphosphates (ddNTP)). In accordance with the present invention, addition of a blocking nucleotide terminates addition of subsequent nucleotides to the 3′-end of the ssDNA fragments. For example, addition of an unextendable ddNTP to the ssDNA fragments would terminate 3′-tail extension by the terminal transferase because ddNTPs lack the 3′-hydroxyl group necessary for addition of a subsequent nucleotide. In one example, the poly-dNTP tail is a poly-G tail that is added to the 3′ ends of the ssDNA fragments in a terminal transferase reaction using a mixture of deoxyguanosine triphosphates (dGTP) and dideoxyguanosine triphosphates (ddGTP). In another example, the poly-dNTP tail is a poly-A tail that is added to the 3′ ends of the ssDNA molecules in a terminal transferase reaction using a mixture of deoxyadenosine triphosphates (dATP) and dideoxyadenosine triphosphates (ddATP). In yet another example, the poly-dNTP tail is a poly-C tail that is added to the 3′ ends of the ssDNA molecules in a terminal transferase reaction using a mixture of deoxycytidine triphosphates (dCTP) and dideoxycytidine triphosphates (ddCTP). In yet another example, the poly-dNTP tail is a poly-T tail that is added to the 3′ ends of the ssDNA molecules in a terminal transferase reaction using a mixture of deoxythymidine triphosphates (dTTP) and dideoxythymidine triphosphates (ddTTP). In one embodiment, the length of the 3′-tail is determined by the amount or concentration of ddNTP in the dNTP and ddNTP reaction mixture used for terminal transferase extension. For example, a mixture of dGTP and ddGTP comprises about 10% ddGTP is used to control the length of the poly-dGTP tail to be about 10 bases on average (i.e., on average one out of every ten nucleotide bases added to the 3′-tail by the terminal transferase is a ddGTP thereby terminating extension of the 3′-tail). In another embodiment, the amount or concentration of ddNTPs (e.g., ddGTP, ddATP, ddCTP and/or ddTTP) comprises about 5% to about 50%, from about 5% to about 25%, or from about 10% to about 20%, of the total deoxynucleotides and dideoxynucleotides (dNTP and ddNTP) included in the reaction mixture. In another embodiment, the amount or concentration of ddNTPs (e.g., ddGTP, ddATP, ddCTP and/or ddTTP) comprises at least 5%, at least 10%, at least 15%, at least 20%, or at least 25%, of the total deoxynucleotides and dideoxynucleotides included in the reaction mixture.

At step 120, a single-stranded DNA (ssDNA) oligonucleotide adapter is hybridized to the 3′-polynucleotide tail of the ssDNA fragment to generate partially double-stranded DNA fragment-adapter constructs. In accordance with the present invention, the adapter comprises a 3′-polynucleotide tail with a complementary sequence to the 3′-polynucleotide tail of the ssDNA fragment. For example, in one embodiment, a poly-G tail is added to the 3′-end of the ssDNA molecules at step 115 and the adapter comprises a complementary poly-C 3′-tail.

In one embodiment, the ssDNA adapters may further comprise a unique molecular identifier (UMI) sequence. As is well known in the art, unique sequence tags (e.g., unique molecular identifiers (UMIs)) can be used to identify unique nucleic acid sequences from a cell-free nucleic acid sample. For example, differing unique sequence tags (UMIs) can be used to differentiate various unique nucleic acid sequence fragments originating from the test sample. In another embodiment, unique sequence tags (UMIs) can be used to reduce amplification bias, which is the asymmetric amplification of different targets due to differences in nucleic acid composition (e.g., high GC content). The unique sequence tags (UMIs) can also be used to discriminate between nucleic acid mutations that arise during amplification. In one embodiment, the unique sequence tag can comprise a short oligonucleotide sequence having a length of from about 2 nt to about 100 nt, from about 2 nt to about 60 nt, from about 2 to about nt, or from about 2 to about 20 nt. In another embodiment, the UMI tag may comprise a short oligonucleotide sequence greater than 5, 6, 7, 8, 9, 10, 11, 12, 13, 14, 15, 16, 17, or 18 nucleotides (nt) in length.

In still another embodiment, the ssDNA adapters utilized in the practice of this invention may include a universal primer and/or one or more sequencing oligonucleotides for use in subsequent cluster generation and/or sequencing (e.g., known P5 and P7 sequences for used in sequencing by synthesis (SBS) (Illumina, San Diego, CA)).

At step 125, the 3′-end of the adapters are extended using a DNA polymerase, and the ssDNA fragment as a template, to generate a plurality of double-stranded DNA (dsDNA) molecules. For example, a DNA polymerase can be used to synthesize, from the free 3′-ends of the adapter, a nucleic acid sequence complementary to the ssDNA fragment. In general, any DNA polymerase can be used in the step. For example, in one embodiment, the extension reaction uses Klenow fragment (3′ to 5′-exo) that is able to read through uracil residues (indicated by white stars) in the converted ssDNA template strand (i.e., ssDNA molecule 215) incorporating adenine (indicated by gray stars) for each uracil. In other embodiments, the polymerase used in the practice of the present invention can be Bst 2.0 (New England BioLabs, Ipswich, MA), Dpo4 (Dpo4), T4 DNA polymerase (T4 DNA polymerase), or DNA polymerase I (New England BioLabs, Ipswich, MA).

At step 130, a double-strand DNA adapter is ligated to the dsDNA molecules obtained from step 125 to generate a plurality of dsDNA adapter-fragment constructs. In accordance with the present invention, the double-strand DNA adapters are ligated to the end of the dsDNA molecules opposite the ssDNA adapter. Optionally, in another embodiment, dsDNA adapters can be ligated to both ends of the dsDNA molecules obtains from step 125 to generate a plurality of dsDNA adapter-fragment constructs. The ligation reaction can be performed using any suitable ligase enzyme which joins the dsDNA adapters to the dsDNA fragments to form dsDNA adapter-fragment constructs. In one example, the ligation reaction is performed using T4 DNA ligase. In another example, T7 DNA ligase is used for adapter ligation to the modified nucleic acid molecule.

In one embodiment, the ends of dsDNA molecules are repaired using, for example, T4 DNA polymerase and Klenow polymerase and phosphorylated with a polynucleotide kinase enzyme. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

Like the ssDNA adapters described above, the dsDNA adapters may comprise a unique molecular identifier (UMI) sequence. The unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag may comprise a short oligonucleotide sequence greater than 5, 6, 7, 8, 9, 10, 11, 12, 13, 14, 15, 16, 17, or 18 nucleotides (nt) in length. Also like the ssDNA adapters described above, the dsDNA adapters may include a universal primer and/or one or more sequencing oligonucleotides for use in subsequent cluster generation and/or sequencing (e.g., known P5 and P7 sequences for used in sequencing by synthesis (SBS) (Illumina, San Diego, CA)).

At step 135, the dsDNA adapter-fragment constructs are amplified to generate a sequencing library. For example, the adapter-fragment dsDNA constructs can be amplified by PCR using a DNA polymerase and a reaction mixture containing primers and a plurality of dNTPs.

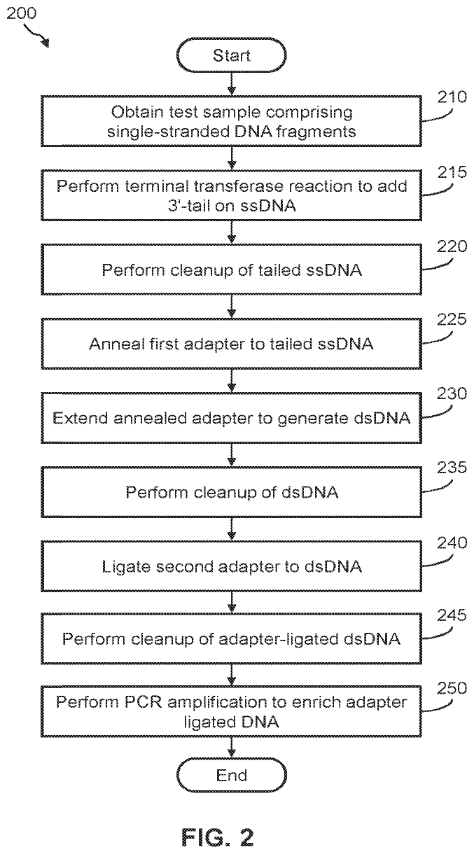

FIG. 2 illustrates a flow diagram of a method 200 for preparing a sequencing library from ssDNA molecules or fragments, in accordance with another embodiment of the present invention. Method 200 includes, but is not limited to, the following steps.

At step 210, a biological test sample is obtained comprising a plurality of ssDNA fragments. As described above, the biological test sample may be a sample selected from the group consisting of whole blood, a blood fraction, plasma, serum, urine, saliva samples, a tissue biopsy, pleural fluid, pericardial fluid, cerebral spinal fluid, and peritoneal fluid. In one embodiments, the biological test sample comprises a plurality of cell-free nucleic acids (e.g., cell-free DNA (cfDNA)) fragments originating from healthy cells and from cancer cells. In another embodiment, the ssDNA sample is a bisulfite-converted DNA sample for methylation profiling of cfDNA. As previously discussed, cell-free nucleic acids (e.g., cfDNA) can be extracted and/or purified from the biological test sample before proceeding with subsequent library preparation steps.

At a step 215, a polynucleotide tail is added to the 3′ ends of the ssDNA fragments. For example, terminal transferase (TdT) and a mixture of a dNTP and a blocking nucleotide (e.g., ddNTP) is used to add a poly-dNTP tail to the 3′ ends of the ssDNA fragments. The blocking nucleotide (e.g., ddNTP) is used to terminate the 3′-tail extension by the terminal transferase. In one example, as previously described, the poly-dNTP tail is a poly-G tail that is added to the 3′ ends of the ssDNA molecules in a terminal transferase reaction using a mixture of dGTP and ddGTP. In another example, the poly-dNTP tail is a poly-A tail that is added to the 3′ ends of the ssDNA molecules in a terminal transferase reaction using a mixture of dATP and ddATP. In yet another example, the poly-dNTP tail is a poly-C tail that is added to the 3′ ends of the ssDNA molecules in a terminal transferase reaction using a mixture of dCTP and ddCTP. In yet another example, the poly-dNTP tail is a poly-T tail that is added to the 3′ ends of the ssDNA molecules in a terminal transferase reaction using a mixture of dTTP and ddTTP. The length of the 3′-tail is determined by the percentage or concentration of ddNTP in the reaction mixture of dNTP and ddNTP used in the terminal transferase reaction. In one example, a mixture of dNTP and ddNTP comprises about 10% ddGTP to control the length of the poly-dNTP tail to be about 10 bases on average. In one embodiment, the amount or concentration of ddNTPs (e.g., ddGTP, ddATP, ddCTP and/or ddTTP) comprises about 5% to about 50%, from about 5% to about 25%, or from about 10% to about 20%, of the total deoxynucleotides and dideoxynucleotides (dNTP and ddNTP) included in the reaction mixture. In another embodiment, the amount or concentration of ddNTPs (e.g., ddGTP, ddATP, ddCTP and/or ddTTP) comprises at least 5%, at least 10%, at least 15%, at least 20%, or at least 25%, of the total deoxynucleotides and dideoxynucleotides included in the reaction mixture.

At a step 220, an optional cleanup step or protocol can be performed on the 3′-tailed ssDNA sample from step 215. The cleanup protocol (e.g., a bead-based protocol) can be utilized, for example, to exchange the existing reaction buffer to a reaction buffer suitable for subsequent synthesis of a second DNA strand using a polymerase. In one example, the cleanup protocol is an SPRI bead based protocol (e.g., 1.8× SPRI; AMPure, Beckman Coulter). As shown in FIG. 2, step 225, a single-stranded DNA (ssDNA) adapter is hybridized to the tailed ssDNA fragments. In one embodiment, the adapter includes, for example, an extendable 3′-OH anchor nucleotide with a sequence that is complementary to the poly-dNTP tail (or a portion thereof) added to the ssDNA fragments. The ssDNA adapters utilized in the practice of this invention may include a unique molecular identifier (UMI), a universal primer and/or one or more sequencing oligonucleotides for use in subsequent cluster generation and/or sequencing (e.g., known P5 and P7 sequences for used in sequencing by synthesis (SBS) (Illumina, San Diego, CA)), as previously described.

In one embodiment, the ssDNA adapter may include a 5′-end biotin label that can be used for subsequent processing steps. For example, the 5′-end biotin label may be used for immobilization or isolation of the ssDNA adapter molecule using streptavidin coated capture beads Immobilization of biotinylated molecules onto streptavidin capture beads can be used to capture all nucleic acid constructs obtained from the ssDNA adapter (e.g., subsequent dsDNA molecules from extension of the adapter, as described herein). In one embodiment, the use of streptavidin coated capture beads allows for isolation of biotin labeled nucleic acid molecules for subsequent cleanup or washing steps, described elsewhere in this application.

At a step 230, the 3′-end of the adapters are extended using a DNA polymerase, and the ssDNA fragment as a template, to generate a plurality of double-stranded DNA (dsDNA) molecules. For example, a DNA polymerase can be used to synthesize, from the free 3′-ends (of the adapter, a nucleic acid sequence complementary to the ssDNA fragment. In general, as described above, any DNA polymerase can be used in the step. In one example, the extension reaction uses a DNA polymerase that generates a blunt-ended extension product.

At step 235, a second optional cleanup protocol (e.g., 1.8×SPRI cleanup protocol) is performed on the dsDNA sample from step 230 to exchange the existing reaction buffer to a reaction buffer suitable for subsequent processing.

At step 240, a double-strand DNA adapter is ligated to the dsDNA molecules obtained from step 235 to generate a plurality of dsDNA adapter-fragment constructs. In accordance with the present invention, the double-strand DNA adapters are ligated to the end of the dsDNA molecules opposite the ssDNA adapter. Optionally, in another embodiment, dsDNA adapters can be ligated to both ends of the dsDNA molecules obtains from step 125 to generate a plurality of dsDNA adapter-fragment constructs. The ligation reaction can be performed using any suitable ligase enzyme which joins the dsDNA adapters to the dsDNA fragments to form dsDNA adapter-fragment constructs. In one example, the ligation reaction is performed using T4 DNA ligase. In another example, T7 DNA ligase is used for adapter ligation to the modified nucleic acid molecule.

As described elsewhere, the dsDNA adapters may comprise a unique molecular identifier (UMI) sequence. The dsDNA adapters may also include a universal primer and/or one or more sequencing oligonucleotides for use in subsequent cluster generation and/or sequencing (e.g., known P5 and P7 sequences for used in sequencing by synthesis (SBS) (Illumina, San Diego, CA)).

In one embodiment, the ends of dsDNA molecules are repaired using, for example, T4 DNA polymerase and Klenow polymerase and phosphorylated with a polynucleotide kinase enzyme. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 245, an optional third cleanup protocol (e.g., 1.8×SPRI cleanup protocol) is performed on the adapter-ligated dsDNA sample from step 240 to exchange the existing reaction buffer to a reaction buffer suitable for subsequent processing. Other cleanup protocols well known in the art may also be used.

At step 250, the dsDNA adapter-fragment constructs are amplified to generate a sequencing library. For example, the adapter-fragment constructs can be amplified by PCR using a DNA polymerase and a reaction mixture containing primers and a plurality of dNTPs.

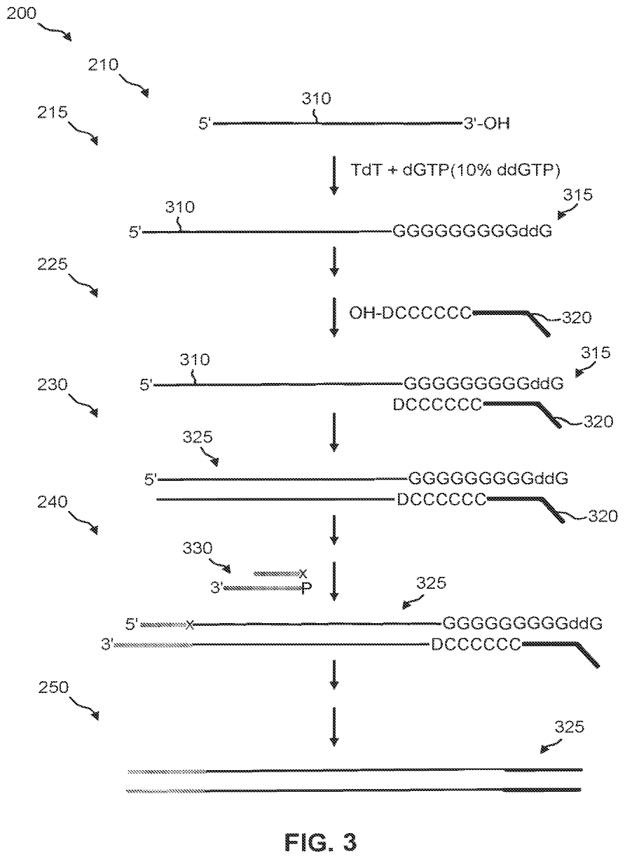

FIG. 3 shows pictorially some of the steps of method 200 of FIG. 2. Namely, at step 210, a ssDNA sample is obtained. In one embodiment, the biological test sample may be a plasma, serum, urine, saliva samples, or tissue biopsy comprising a plurality of cell-free nucleic acids (e.g., cell-free DNA (cfDNA)) fragments originating from healthy cells and from cancer cells, as described above. In another embodiment, the ssDNA sample is a bisulfite-converted DNA sample for methylation profiling of cfDNA, which is represented by a ssDNA molecule 310.

At step 215, a 3′-tail 315 is added to the 3′-OH ends of ssDNA fragments 310. For example, terminal transferase (TdT) and a mixture of dGTP and ddGTP is used to add a dGTP tail to the 3′-end of ssDNA molecule 310. Addition of a blocking ddGTP nucleotide terminates the 3′-tail extension by the terminal transferase. In one embodiment, a mixture of dGTP and ddGTP comprises about 10% ddGTP to control the length of the poly-dNTP tail to be about 10 bases on average (as described above). In other embodiments, the ddGTP concentration can be at least 5%, at least 10%, at least 15%, at least 20%, or at least 25% of the total amount of deoxyguanosine triphosphates (dGTP) and dideoxyguanosine triphosphates (ddGTP) included in the reaction mixture

At step 225 (step 220 is not shown), a first adapter 320 is hybridized to the 3′-tail 315 on ssDNA fragment 310. First adapter 320 includes, for example, a 3′-OH (indicated by “D”) nucleotide, a poly-dCTP sequence that is complementary to a portion of 3′-tail 315 on ssDNA fragment 310, and a universal primer sequence. The free 3′-OH anchor nucleotide D is used to anchor the hybridization of first adapter 320 to the beginning of 3′-tail 315 on ssDNA molecule 310.

At step 230, adapter-annealed ssDNA molecule 310 is converted to a dsDNA molecule 325 in an extension reaction. For example, a DNA polymerase is used to extend first adapter 320 from the 3′-OH of the anchor nucleotide (D). In one example, the extension reaction uses a DNA polymerase that generates a blunt-ended extension product.

At step 240 (step 235 is not shown), a second adapter 330 is added to dsDNA 325 using a dsDNA ligation reaction. Second adapter 330 may include, for example, a 3′ blocked nucleotide (indicated by “x”) to prevent the formation of adapter dimers, a 5′ phosphorylated nucleotide (indicated by “P”) for ligation and a universal primer sequence.

At step 250 (step 145 is not shown), adapter-dsDNA construct 325 is enriched for sequencing using PCR amplification. For example, amplification primers (not shown) that include (among other sequences) sequences that are complementary to the universal primer sequences in first adapter 320 and second adapter 330 are used in the amplification reaction to amplify and enrich adapter-dsDNA constructs preparing a library for subsequent sequencing.

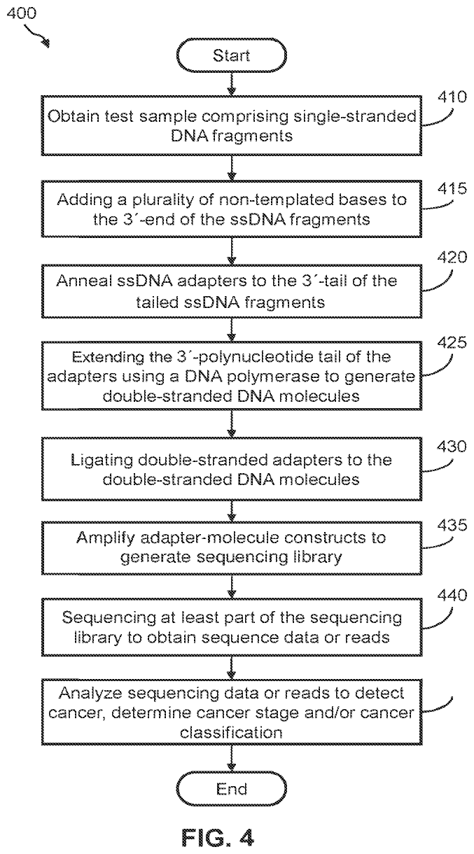

FIG. 4 is a flow diagram illustrating a method 400 for preparing a sequencing library from a cell-free DNA test sample for use in detecting cancer, determining cancer status, monitoring cancer progression, and/or determining a cancer classification.

As shown in FIG. 4, at step 410, a biological test sample is obtained from a subject (e.g., a patient) known to have or suspected of having cancer. In one embodiment, the biological test sample may be a sample selected from the group consisting of blood, plasma, serum, urine and saliva samples. In other embodiments, the sample is a plasma sample from a cancer patient, or a patient suspected of having cancer. Alternatively, as noted above, the biological sample may comprise a sample selected from the group consisting of whole blood, a blood fraction, a tissue biopsy, pleural fluid, pericardial fluid, cerebral spinal fluid, and peritoneal fluid. In accordance with some embodiments, the biological test sample comprises a plurality of cell-free nucleic acids (e.g., cell-free DNA (cfDNA)) fragments originating from healthy cells and from cancer cells. Optionally, in one embodiment, cell-free nucleic acids (e.g., cfDNA) can be extracted and/or purified from the biological test sample before proceeding with subsequent library preparation steps. In general, any known method in the art can be used to extract and purify cell-free nucleic acids from the biological test sample. For example, cell-free nucleic acids can be extracted and purified using one or more known commercially available protocols or kits, such as the QIAamp circulating nucleic acid kit (Qiagen) or MagMAX Cell-Free DNA Isolation kit (Thermo Fisher Scientific).

At step 415, a polynucleotide tail is added to the 3′ ends of the ssDNA molecules. For example, terminal transferase (TdT) and a reaction mixture of a deoxynucleotide triphosphates (dNTP) is used to add a poly-dNTP tail to the 3′-ends of the ssDNA fragments. In one embodiment, the reaction mixture may further comprise one or more blocking nucleotide (e.g., one or more dideoxynucleotide triphosphates (ddNTP)). In accordance with the present invention, addition of a blocking nucleotide terminates addition of nucleotides to the 3′-end of the ssDNA fragments. For example, addition of an unextendable ddNTP to the ssDNA fragments would terminate 3′-tail extension by the terminal transferase as ddNTPs lack an extendable 3′-OH tail. In one example, the poly-dNTP tail is a poly-G tail that is added to the 3′ ends of the ssDNA fragments in a terminal transferase reaction using a mixture of deoxyguanosine triphosphates (dGTP) and dideoxyguanosine triphosphates (ddGTP). In another example, the poly-dNTP tail is a poly-A tail that is added to the 3′ ends of the ssDNA molecules in a terminal transferase reaction using a mixture of deoxyadenosine triphosphates (dATP) and dideoxyadenosine triphosphates (ddATP). In yet another example, the poly-dNTP tail is a poly-C tail that is added to the 3′ ends of the ssDNA molecules in a terminal transferase reaction using a mixture of deoxycytidine triphosphates (dCTP) and dideoxycytidine triphosphates (ddCTP). In yet another example, the poly-dNTP tail is a poly-T tail that is added to the 3′ ends of the ssDNA molecules in a terminal transferase reaction using a mixture of deoxythymidine triphosphates (dTTP) and dideoxythymidine triphosphates (ddTTP). In one embodiment, the length of the 3′-tail is determined by the amount or concentration of ddNTP in the dNTP and ddNTP reaction mixture used for terminal transferase extension. For example, a mixture of dGTP and ddGTP comprises about 10% ddGTP is used to control the length of the poly-dGTP tail to be about 10 bases on average (i.e., on average one out of every ten nucleotide bases added to the 3′-tail by the terminal transferase is a ddGTP thereby terminating extension of the 3′-tail). In other embodiments, the amount or concentration of ddNTPs (e.g., ddGTP, ddATP, ddCTP and/or ddTTP) comprises about 5% to about 50%, from about 5% to about 25%, or from about 10% to about 20%, of the total deoxynucleotides and dideoxynucleotides (dNTP and ddNTP) included in the reaction mixture. In another embodiment, the amount or concentration of ddNTPs (e.g., ddGTP, ddATP, ddCTP and/or ddTTP) comprises at least 5%, at least 10%, at least 15%, at least 20%, or at least 25%, of the total deoxynucleotides and dideoxynucleotides included in the reaction mixture.

At step 420, a single-stranded DNA (ssDNA) oligonucleotide adapter is hybridized to the 3′-polynucleotide tail of the ssDNA fragment to generate partially double-stranded DNA fragment-adapter constructs. In accordance with the present invention, the adapter comprises a 3′-polynucleotide tail with a complementary sequence to the 3′-polynucleotide tail of the ssDNA fragment. For example, in one embodiment, a poly-G tail is added to the 3′-end of the ssDNA molecules at step 415 and the adapter comprises a complementary poly-C 3′-tail.

The ssDNA adapters utilized in the practice of this invention may include a unique molecular identifier (UMI), a universal primer and/or one or more sequencing oligonucleotides for use in subsequent cluster generation and/or sequencing (e.g., known P5 and P7 sequences for used in sequencing by synthesis (SBS) (Illumina, San Diego, CA)), as previously described.

In one embodiment, the ssDNA adapter may include a 5′-end biotin label that can be used for subsequent processing steps. For example, the 5′-end biotin label may be used for immobilization or isolation of the ssDNA adapter molecule using streptavidin coated capture beads Immobilization of biotinylated molecules onto streptavidin capture beads can be used to capture all nucleic acid constructs obtained from the ssDNA adapter (e.g., subsequent dsDNA molecules from extension of the adapter, as described herein). In one embodiment, the use of streptavidin coated capture beads allows for isolation of biotin labeled nucleic acid molecules for subsequent cleanup or washing steps, described elsewhere in this application.

At step 425, the 3′-end of the adapters are extended using a DNA polymerase, and the ssDNA fragment as a template, to generate a plurality of double-stranded DNA (dsDNA) molecules. For example, a DNA polymerase can be used to synthesize, from the free 3′-ends (of the adapter, a nucleic acid sequence complementary to the ssDNA fragment. In general, as described above, any DNA polymerase can be used in the step.

At step 430, a double-strand DNA adapter is ligated to the dsDNA molecules obtained from step 425 to generate a plurality of dsDNA adapter-fragment constructs. In accordance with the present invention, the double-strand DNA adapters are ligated to the end of the dsDNA molecules opposite the ssDNA adapter. Optionally, in another embodiment, dsDNA adapters can be ligated to both ends of the dsDNA molecules obtains from step 125 to generate a plurality of dsDNA adapter-fragment constructs. The ligation reaction can be performed using any suitable ligase enzyme which joins the dsDNA adapters to the dsDNA fragments to form dsDNA adapter-fragment constructs. In one example, the ligation reaction is performed using T4 DNA ligase. In another example, T7 DNA ligase is used for adapter ligation to the modified nucleic acid molecule.

In one embodiment, the ends of dsDNA molecules are repaired using, for example, T4 DNA polymerase and Klenow polymerase and phosphorylated with a polynucleotide kinase enzyme. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

Like the ssDNA adapters described above, the dsDNA adapters may comprise a unique molecular identifier (UMI) sequence. The unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag may comprise a short oligonucleotide sequence greater than 5, 6, 7, 8, 9, 10, 11, 12, 13, 14, 15, 16, 17, or 18 nucleotides (nt) in length. Also like the ssDNA adapters described above, the dsDNA adapters may include a universal primer and/or one or more sequencing oligonucleotides for use in subsequent cluster generation and/or sequencing (e.g., known P5 and P7 sequences for used in sequencing by synthesis (SBS) (Illumina, San Diego, CA)).

At step 435, the dsDNA adapter-fragment constructs are amplified to generate a sequencing library. For example, the adapter-adapter constructs can be amplified by PCR using a DNA polymerase and a reaction mixture containing primers and a plurality of dNTPs.

At step 440 at least a portion of sequence library obtained from step 435 is sequenced to obtain sequencing data or sequence reads. In general, any method known in the art can be used to obtain sequence data or sequence reads from a test sample. For example, in one embodiment, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step is performed prior to sequencing. In certain embodiments, the sequencing comprises whole genome sequencing (or shotgun sequencing) of the cfDNA library to provide sequence data or sequencing reads representative of a whole genome. In other embodiments, the sequencing comprises targeted sequencing of the cfDNA library. For example, the sequencing library can be enriched for specific target sequences (e.g., using a plurality of hybridization probes to pull down cfDNA fragments known to be, or suspected of being, indicative of cancer) and the targeted sequences sequenced.

At step 450, the sequencing data or sequencing reads can be analyzed for detecting the presence of absence of cancer, determining cancer stage, monitoring cancer progression, and/or for determining a cancer classification (e.g., cancer type or cancer tissue of origin). In another embodiment, the sequencing data or reads can be used to infer the presence or absence of cancer, cancer status, and/or a cancer classification. For example, the sequencing data or sequencing reads can be analyzed to identify methylation profiles indicative of the presence or absence of cancer (see, e.g., PCT Application No. PCT/AU2013/001088, filed Sep. 20, 2013, now WO 2014/043763 A1) or to identify one or more mutational signatures indicative of the presence or absence of cancer (see, e.g., PCT Application No. PCT/US2017/060472, filed Nov. 7, 2017). In other embodiments, the sequence data or sequence reads can be analyzed to assess the fractional contribution of different tissues to a DNA mixtures (e.g., for assessment of a cancer tissue of origin) as described in PCT Application No. PCT/CN2015/084442, filed Jul. 20, 2015, now WO 2016/008451. Alternatively, the sequencing data or sequencing reads can be utilized to analyze nucleic acid fragmentation patterns for the detection and/or classification of cancer (e.g., cancer tissue of origin) as described in PCT Application No. PCT/CN2016/091531, filed Jul. 25, 2016, now WO 2017/012592.

In one embodiment, the sequencing data or sequencing reads can be analyzed to detect the presence or absence of, determine the stage of, monitor progression of, and/or classify a carcinoma, a sarcoma, a myeloma, a leukemia, a lymphoma, a blastoma, a germ cell tumor, or any combination thereof. In some embodiments, the carcinoma may be an adenocarcinoma. In other embodiments, the carcinoma may be a squamous cell carcinoma. In still other embodiments, the carcinoma is selected from the group consisting of: small cell lung cancer, non-small-cell lung, nasopharyngeal, colorectal, anal, liver, urinary bladder, cervical, testicular, ovarian, gastric, esophageal, head-and-neck, pancreatic, prostate, renal, thyroid, melanoma, and breast carcinoma. In another embodiment, the sequencing data or sequencing reads can be analyzed to detect presence or absence of, determine the stage of, monitor progression of, and/or classify a sarcoma. In certain embodiments, the sarcoma can be selected from the group consisting of: osteosarcoma, chondrasarcoma, leiomyosarcoma, rhabdomyosarcoma, mesothelial sarcoma (mesothelioma), fibrosarcoma, angiosarcoma, liposarcoma, glioma, and astrocytoma. In still another embodiment, the sequencing data or sequencing reads can be analyzed to detect presence or absence of, determine the stage of, monitor progression of, and/or classify leukemia. In certain embodiments, the leukemia can be selected from the group consisting of: myelogenous, granulocytic, lymphatic, lymphocytic, and lymphoblastic leukemia. In still another embodiment, the sequencing data or sequencing reads can be used to detect presence or absence of, determine the stage of, monitor progression of, and/or classify a lymphoma. In certain embodiments, the lymphoma can be selected from the group consisting of: Hodgkin's lymphoma and Non-Hodgkin's lymphoma.

Sequencing and Bioinformatics

Aspects of the invention include sequencing of nucleic acid molecules to generate a plurality of sequence reads, and bioinformatic manipulation of the sequence reads to carry out the subject methods.

In certain embodiments, a sample is collected from a subject, followed by enrichment for genetic regions or genetic fragments of interest. For example, in some embodiments, a sample can be enriched by hybridization to a nucleotide array comprising cancer-related genes or gene fragments of interest. In some embodiments, a sample can be enriched for genes of interest (e.g., cancer-associated genes) using other methods known in the art, such as hybrid capture. See, e.g., Lapidus (U.S. Pat. No. 7,666,593), the contents of which is incorporated by reference herein in its entirety. In one hybrid capture method, a solution-based hybridization method is used that includes the use of biotinylated oligonucleotides and streptavidin coated magnetic beads. See, e.g., Duncavage et al., J Mol Diagn. 13(3): 325-333 (2011); and Newman et al., Nat Med. 20(5): 548-554 (2014). Isolation of nucleic acid from a sample in accordance with the methods of the invention can be done according to any method known in the art.

Sequencing may be by any method or combination of methods known in the art. For example, known DNA sequencing techniques include, but are not limited to, classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, Polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977), the contents of which are incorporated by reference herein in their entirety. Another conventional sequencing method involves chemical degradation of nucleic acid fragments. See, Maxam et al., Proc. Natl. Acad. Sci., 74: 560 564 (1977), the contents of which are incorporated by reference herein in their entirety. Methods have also been developed based upon sequencing by hybridization. See, e.g., Harris et al., (U.S. patent application number 2009/0156412), the contents of which are incorporated by reference herein in their entirety.

A sequencing technique that can be used in the methods of the provided invention includes, for example, Helicos True Single Molecule Sequencing (tSMS) (Harris T. D. et al. (2008) Science 320:106-109), the contents of which are incorporated by reference herein in their entirety. Further description of tSMS is shown, for example, in Lapidus et al. (U.S. Pat. No. 7,169,560), the contents of which are incorporated by reference herein in their entirety, Lapidus et al. (U.S. patent application publication number 2009/0191565, the contents of which are incorporated by reference herein in their entirety), Quake et al. (U.S. Pat. No. 6,818,395, the contents of which are incorporated by reference herein in their entirety), Harris (U.S. Pat. No. 7,282,337, the contents of which are incorporated by reference herein in their entirety), Quake et al. (U.S. patent application publication number 2002/0164629, the contents of which are incorporated by reference herein in their entirety), and Braslaysky, et al., PNAS (USA), 100: 3960-3964 (2003), the contents of which are incorporated by reference herein in their entirety.

Another example of a DNA sequencing technique that can be used in the methods of the provided invention is 454 sequencing (Roche) (Margulies, M et al. 2005, Nature, 437, 376-380, the contents of which are incorporated by reference herein in their entirety). Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application publication numbers 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the contents of each of which are incorporated by reference herein in their entirety).

In some embodiments, the sequencing technology is Illumina sequencing. Illumina sequencing is based on the amplification of DNA on a solid surface using fold-back PCR and anchored primers. Genomic DNA can be fragmented, or in the case of cfDNA, fragmentation is not needed due to the already short fragments. Adapters are ligated to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. Yet another example of a sequencing technique that can be used in the methods of the provided invention is nanopore sequencing (Soni G V and Meller A. (2007) Clin Chem 53: 1996-2001, the contents of which are incorporated by reference herein in their entirety). Another example of a sequencing technique that can be used in the methods of the provided invention involves using a chemical-sensitive field effect transistor (chemFET) array to sequence DNA (for example, as described in US Patent Application Publication No. 20090026082, the contents of which are incorporated by reference herein in their entirety). Another example of a sequencing technique that can be used in the methods of the provided invention involves using an electron microscope (Moudrianakis E. N. and Beer M. Proc Natl Acad Sci USA. 1965 March; 53:564-71, the contents of which are incorporated by reference herein in their entirety).

If the nucleic acid from the sample is degraded or only a minimal amount of nucleic acid can be obtained from the sample, PCR can be performed on the nucleic acid in order to obtain a sufficient amount of nucleic acid for sequencing (See, e.g., Mullis et al. U.S. Pat. No. 4,683,195, the contents of which are incorporated by reference herein in its entirety).

### Biological Samples

Aspects of the invention involve obtaining a sample, e.g., a biological sample, such as a tissue and/or body fluid sample, from a subject for purposes of analyzing a plurality of nucleic acids (e.g., a plurality of cfDNA molecules) therein. Samples in accordance with embodiments of the invention can be collected in any clinically-acceptable manner Any sample suspected of containing a plurality of nucleic acids can be used in conjunction with the methods of the present invention. In some embodiments, a sample can comprise a tissue, a body fluid, or a combination thereof. In some embodiments, a biological sample is collected from a healthy subject. In some embodiments, a biological sample is collected from a subject who is known to have a particular disease or disorder (e.g., a particular cancer or tumor). In some embodiments, a biological sample is collected from a subject who is suspected of having a particular disease or disorder.

As used herein, the term “tissue” refers to a mass of connected cells and/or extracellular matrix material(s). Non-limiting examples of tissues that are commonly used in conjunction with the present methods include skin, hair, finger nails, endometrial tissue, nasal passage tissue, central nervous system (CNS) tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or non-human mammal Tissue samples in accordance with embodiments of the invention can be prepared and provided in the form of any tissue sample types known in the art, such as, for example and without limitation, formalin-fixed paraffin-embedded (FFPE), fresh, and fresh frozen (FF) tissue samples.

As used herein, the term “body fluid” refers to a liquid material derived from a subject, e.g., a human or non-human mammal. Non-limiting examples of body fluids that are commonly used in conjunction with the present methods include mucous, blood, plasma, serum, serum derivatives, synovial fluid, lymphatic fluid, bile, phlegm, saliva, sweat, tears, sputum, amniotic fluid, menstrual fluid, vaginal fluid, semen, urine, cerebrospinal fluid (CSF), such as lumbar or ventricular CSF, gastric fluid, a liquid sample comprising one or more material(s) derived from a nasal, throat, or buccal swab, a liquid sample comprising one or more materials derived from a lavage procedure, such as a peritoneal, gastric, thoracic, or ductal lavage procedure, and the like.

In some embodiments, a sample can comprise a fine needle aspirate or biopsied tissue. In some embodiments, a sample can comprise media containing cells or biological material. In some embodiments, a sample can comprise a blood clot, for example, a blood clot that has been obtained from whole blood after the serum has been removed. In some embodiments, a sample can comprise stool. In one preferred embodiment, a sample is drawn whole blood. In one aspect, only a portion of a whole blood sample is used, such as plasma, red blood cells, white blood cells, and platelets. In some embodiments, a sample is separated into two or more component parts in conjunction with the present methods. For example, in some embodiments, a whole blood sample is separated into plasma, red blood cell, white blood cell, and platelet components.

In some embodiments, a sample includes a plurality of nucleic acids not only from the subject from which the sample was taken, but also from one or more other organisms, such as viral DNA/RNA that is present within the subject at the time of sampling.

Nucleic acid can be extracted from a sample according to any suitable methods known in the art, and the extracted nucleic acid can be utilized in conjunction with the methods described herein. See, e.g., Maniatis, et al., Molecular Cloning: A Laboratory Manual, Cold Spring Harbor, N.Y., pp. 280-281, 1982, the contents of which are incorporated by reference herein in their entirety.

In one preferred embodiment, cell free nucleic acid (e.g., cfDNA) is extracted from a sample. cfDNA are short base nuclear-derived DNA fragments present in several bodily fluids (e.g. plasma, stool, urine). See, e.g., Mouliere and Rosenfeld, PNAS 112(11): 3178-3179 (Mar 2015); Jiang et al., PNAS (Mar 2015); and Mouliere et al., Mol Oncol, 8(5):927-41 (2014). Tumor-derived circulating tumor DNA (ctDNA) constitutes a minority population of cfDNA, in some cases, varying up to about 50%. In some embodiments, ctDNA varies depending on tumor stage and tumor type. In some embodiments, ctDNA varies from about 0.001% up to about 30%, such as about 0.01% up to about 20%, such as about 0.01% up to about 10%. The covariates of ctDNA are not fully understood, but appear to be positively correlated with tumor type, tumor size, and tumor stage. E.g., Bettegowda et al, Sci Trans Med, 2014; Newmann et al, Nat Med, 2014. Despite the challenges associated with the low population of ctDNA in cfDNA, tumor variants have been identified in ctDNA across a wide span of cancers. E.g., Bettegowda et al, Sci Trans Med, 2014. Furthermore, analysis of cfDNA versus tumor biopsy is less invasive, and methods for analyzing, such as sequencing, enable the identification of sub-clonal heterogeneity. Analysis of cfDNA has also been shown to provide for more uniform genome-wide sequencing coverage as compared to tumor tissue biopsies. In some embodiments, a plurality of cfDNA is extracted from a sample in a manner that reduces or eliminates co-mingling of cfDNA and genomic DNA. For example, in some embodiments, a sample is processed to isolate a plurality of the cfDNA therein in less than about 2 hours, such as less than about 1.5, 1 or 0.5 hours.

A non-limiting example of a procedure for preparing nucleic acid from a blood sample follows. Blood may be collected in 10 mL EDTA tubes (for example, the BD VACUTAINER® family of products from Becton Dickinson, Franklin Lakes, New Jersey), or in collection tubes that are adapted for isolation of cfDNA (for example, the CELL FREE DNA BCT® family of products from Streck, Inc., Omaha, Nebraska) can be used to minimize contamination through chemical fixation of nucleated cells, but little contamination from genomic DNA is observed when samples are processed within 2 hours or less, as is the case in some embodiments of the present methods. Beginning with a blood sample, plasma may be extracted by centrifugation, e.g., at 3000 rpm for 10 minutes at room temperature minus brake. Plasma may then be transferred to 1.5 ml tubes in 1 ml aliquots and centrifuged again at 7000 rpm for 10 minutes at room temperature. Supernatants can then be transferred to new 1.5 ml tubes. At this stage, samples can be stored at −80° C. In certain embodiments, samples can be stored at the plasma stage for later processing, as plasma may be more stable than storing extracted cfDNA.

Plasma DNA can be extracted using any suitable technique. For example, in some embodiments, plasma DNA can be extracted using one or more commercially available assays, for example, the QIAmp Circulating Nucleic Acid Kit family of products (Qiagen N.V., Venlo Netherlands). In certain embodiments, the following modified elution strategy may be used. DNA may be extracted using, e.g., a QIAmp Circulating Nucleic Acid Kit, following the manufacturer's instructions (maximum amount of plasma allowed per column is 5 mL). If cfDNA is being extracted from plasma where the blood was collected in Streck tubes, the reaction time with proteinase K may be doubled from 30 min to 60 min Preferably, as large a volume as possible should be used (i.e., 5 mL). In various embodiments, a two-step elution may be used to maximize cfDNA yield. First, DNA can be eluted using 30 μL of buffer AVE for each column. A minimal amount of buffer necessary to completely cover the membrane can be used in the elution in order to increase cfDNA concentration. By decreasing dilution with a small amount of buffer, downstream desiccation of samples can be avoided to prevent melting of double stranded DNA or material loss. Subsequently, about 30 μL of buffer for each column can be eluted. In some embodiments, a second elution may be used to increase DNA yield.

### Computer Systems and Devices

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processors of any kind of digital computer. Generally, a processor will receive instructions and data from a read-only memory or a random access memory, or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. For example, a reference set of data may be stored at a remote location and a computer can communicate across a network to access the reference data set for comparison purposes. In other embodiments, however, a reference data set can be stored locally within the computer, and the computer accesses the reference data set within the CPU for comparison purposes. Examples of communication networks include, but are not limited to, cell networks (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, a data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium that can be used to store the desired information, and which can be accessed by a computing device.

Functions described herein can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the invention, a computer system for implementing some or all of the described inventive methods can include one or more processors (e.g., a central processing unit (CPU) a graphics processing unit (GPU), or both), main memory and static memory, which communicate with each other via a bus.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to, one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the invention can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database included in a system. Any suitable database structure may be used including relational databases, object-oriented databases, and others. In some embodiments, reference data is stored in a relational database such as a “not-only SQL” (NoSQL) database. In certain embodiments, a graph database is included within systems of the invention. It is also to be understood that the term “database” as used herein is not limited to one single database; rather, multiple databases can be included in a system. For example, a database can include two, three, four, five, six, seven, eight, nine, ten, fifteen, twenty, or more individual databases, including any integer of databases therein, in accordance with embodiments of the invention. For example, one database can contain public reference data, a second database can contain test data from a patient, a third database can contain data from healthy subjects, and a fourth database can contain data from sick subjects with a known condition or disorder. It is to be understood that any other configuration of databases with respect to the data contained therein is also contemplated by the methods described herein.

References and citations to other documents, such as patents, patent applications, patent publications, journals, books, papers, web contents, have been made throughout this disclosure. All such documents are hereby incorporated herein by reference in their entirety for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof. All references cited throughout the specification are expressly incorporated by reference herein.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

What is claimed is:

1. A method for preparing a sequencing library from a test sample comprising a plurality of single-strand DNA fragments, the method comprising:
(a) obtaining a test sample comprising a plurality of single-stranded DNA (ssDNA) fragments;
(b) adding a plurality of non-templated nucleotide bases to the 3′-end of the ssDNA fragments generating a plurality of 3′-polynucleotide tailed ssDNA fragments;
(c) annealing a plurality of single-stranded DNA (ssDNA) oligonucleotide adapters to the 3′-end of the polynucleotide tailed ssDNA fragment to generate a partially double-stranded DNA fragment-adapter constructs, wherein the oligonucleotide adapters comprises a region complementary to the 3′-polynucleotide tail of the tailed ssDNA fragments;
(d) extending the 3′-tail of the oligonucleotide adapters using a DNA polymerase and the ssDNA fragment as a template to generate a plurality of double-stranded DNA (dsDNA) molecules;
(e) ligating a double-strand DNA adapter to the plurality of dsDNA molecules obtained from step (d) to generate a plurality of dsDNA adapter-molecule constructs, wherein the double-strand DNA adapters are ligated to the end of the dsDNA opposite the ssDNA oligonucleotide adapters; and
(f) amplifying the dsDNA adapter-molecule constructs to generate a sequencing library.

2. The method according to claim 1, wherein the addition of non-templated bases to the 3′-end of the ssDNA fragments in step (b) is catalyzed using a terminal transferase and a reaction mixture comprising one or more dNTPs.

3. The method according to claim 2, wherein the terminal transferase reaction mixture comprises dGTPs and a 3′-polyguanine (poly-G) tail is added in step (b) to the ssDNA fragments.

4. The method according to claim 3, wherein the terminal transferase reaction mixture further comprises a blocking nucleotide, and wherein incorporation of the blocking nucleotide terminates 3′-tail extension by the terminal transferase.

5. The method according to claim 4, wherein the blocking nucleotide is ddGTPs.

6. The method according to claim 5, wherein the ddGTPs comprises at least 5% of the total nucleotides included in the reaction mix.

7. The method according to claim 5, wherein the ddGTPs comprises at least 10% of the total nucleotides included in the reaction mix.

8. The method according to claim 5, wherein the ddGTPs comprises at least 20% of the total nucleotides included in the reaction mix.

9. The method according to any preceding claim, wherein the ssDNA oligonucleotide adapter includes a 5′-end biotin label.

10. The method according to claim 9, wherein the plurality of 5′-end biotin labeled ssDNA oligonucleotide adapter are isolated using streptavidin-coated beads.

11. The method according to any preceding claim, wherein the plurality of dsDNA molecules generated in step (d) are modified prior to ligation of the dsDNA adapters in step (e).

12. The method according to claim 11, wherein the modification comprises end-repairing, A-tailing, phosphorylation, or any combination thereof.

13. The method according to any preceding claim, wherein the double-strand DNA adapters are ligated to the end of the dsDNA molecules opposite the ssDNA adapter.

14. The method according to any preceding claim, wherein the test sample is from whole blood, a blood fraction, plasma, serum, urine, fecal, saliva, a tissue biopsy, pleural fluid, pericardial fluid, cerebral spinal fluid, or peritoneal fluid.

15. The method according to any preceding claim, wherein the ssDNA fragments are cell-free DNA (cfDNA) fragments.

16. The method according to any preceding claim, wherein the test sample is a plasma sample obtained from a subject known to have, or suspected of having cancer.

17. The method according to any preceding claim, wherein the test sample includes ssDNA fragments originating from healthy cells and from cancer cells.

18. The method according to any preceding claim, wherein the ssDNA fragments are isolated from the test sample, prior to addition of non-templated bases to the 3′-ends in step (b).

19. The method according to any preceding claim, wherein the dsDNA adapter ligated to the dsDNA molecules in added in step (e) is a Y-shaped sequencing adapter.

20. The method according to claim 19, wherein the Y-shaped sequencing adapter is formed by annealing a pair of partially complementary oligonucleotides to one another, wherein the Y-shaped adapter comprises a first double-stranded region, formed from hybridization between the complementary regions of the oligonucleotides, and a second single-stranded region.

21. The method according to any preceding claim, wherein the ligase is T4 DNA ligase.

22. The method according to any preceding claim, wherein the ligase is T7 DNA ligase.

23. The method according to any preceding claim, wherein the method further comprises:
(g) sequencing the library to obtain a plurality of sequence reads; and
(h) detecting the presence of absence of cancer, determining cancer status, monitoring cancer progression and/or determining a cancer classification from the plurality of sequence reads.

24. The method according to any preceding claim, wherein the library is enriched for one or more target dsDNA fragments using hybridization probes to pull down dsDNA fragments known to be, or suspected of being, indicative of cancer and the target sequences sequenced.

25. The method according to any preceding claim, wherein the sequence reads are obtained from next-generation sequencing (NGS).

26. The method according to any preceding claim, wherein the sequence reads are obtained from massively parallel sequencing using sequencing-by-synthesis.

27. The method according to any preceding claim, wherein the sequence reads are obtained from paired-end sequencing.

28. The method according to any preceding claim, wherein monitoring cancer progression further comprises monitoring disease progression, monitoring therapy, or monitoring cancer growth.

29. The method according to any preceding claim, wherein the cancer classification comprises determining cancer type and/or cancer tissue of origin.

30. The method according to any preceding claim, wherein the cancer comprises a carcinoma, a sarcoma, a myeloma, a leukemia, a lymphoma, a blastoma, a germ cell tumor, or any combination thereof.
